(19) United States
(12) Patent Application Publication
(10) Pub. No.: US 2025/0027022 A1
(43) Pub. Date: Jan. 23, 2025

(54) ANAEROBIC DIGESTER WITH STIRRING AND REMOTE MONITORING

(71) Applicant: UNIVERSIDAD PONTIFICIA BOLIVARIANA, Medellin (CO)

(72) Inventors: Sergio Andrés ARANGO OSORIO, Medellin (CO); Carlos Andrés BUSTAMANTE CHAVERRA, Medellin (CO); Jorge Wilson GONZÁLEZ, Medellín (CO); Idi Amin ISAAC MILLAN, Medellin (CO); Gabriel Jaime LÓPEZ JIMÉNEZ, Medellin (CO); Silvio Andrés SALAZAR RAMÍREZ, Medellin (CO); Raúl Adolfo VALENCIA CARDONA, Medellin (CO); Oscar Hernán VASCO ECHEVERRI, Medellin (CO)

(21) Appl. No.: 18/707,565

(22) PCT Filed: Nov. 1, 2022
(86) PCT No.: PCT/IB2022/060534

§ 371 Date: May 3, 2024

(30) Foreign Application Priority Data
Nov. 5, 2021 (CO) NC2021/0014996

(51) Int. Cl. C12M 1/00; B09B 3/65; C05F 17/70; C05F 17/979; C05F 17/993; C12M 1/107; C12M 1/34

(52) U.S. Cl. CPC C12M 29/00; B09B 3/65; C05F 17/70; C05F 17/979; C05F 17/993; C12M 21/04; C12M 29/04; C12M 41/12; C12M 41/40

(57) ABSTRACT

The present invention relates to an organic matter biodigester comprising a tank that has an organic matter inlet, a first connection arranged in an upper portion of the tank and a second connection arranged in the lower portion of the tank. Furthermore, said tank also has a biogas outlet and a lower surface configured to rest on a support surface. Furthermore, the lower portion of the tank is at a shorter distance from the support surface compared to the upper portion of the tank. Said organic matter biodigester also comprises a manual pump with a pump inlet connected in the second connection, and a pump outlet connected in the first connection of the tank. The pump is configured to recirculate the organic matter inside the tank from the second connection towards the first connection; and a porous layer arranged inside the tank.

### TECHNICAL FIELD OF THE INVENTION

The present disclosure relates to fuel production systems. Particularly, the disclosure relates to biodigesters for biogas production.

### BACKGROUND

With the current energy demand and the search for solutions to reduce the impact caused by conventional energy generation sources, biodigesters have been developed consisting of bioreactors or anaerobic digesters to which organic matter dissolved in water is fed. Within the system, an anaerobic fermentation process is carried out with the help of microorganisms, with one of its main products being biogas, which contains methane and traces of other gases. Another of the resulting products is a liquid by-product, which can be used as a fertilizer, since it is rich in inorganic nitrates (NO₃), potassium (K), and phosphorus (P).

This type of technology has great potential for environmental protection as it reduces the amount of waste discharged into ecosystems and also operates as a non-conventional energy alternative. Thus, some documents related to biodigesters are reported in the prior art, such as US20190144304A1 and US2009130704A1.

For instance, document US20190144304A1 discloses a system for the comprehensive treatment of crop contamination in a pig farm. Document US2019014430304A1, in particular, discloses a fermentation reactor with a circulation pump that is, in turn, connected to a boiler. Said circulation pump is also connected to a reactor inlet through a three-way valve that may, in turn, be connected to other reactors.

Document US2009130704A1, for its part, discloses a bioreactor comprising structural elements made of porous materials. The porous materials of US2009130704A1 have open pore structures. US2009130704A1, in particular, indicates that the bioreactor includes a reservoir with an inlet conduit connected with a recirculation pump, wherein a culture medium is circulated through the bioreactor via said pump. On the other hand, it also discloses a nutrient addition port corresponding to a vertical pipe arranged at the upper end of a bioreactor.

### SUMMARY

The present disclosure relates to an organic matter biodigester, comprising a tank having an organic matter inlet, a first connection arranged in an upper portion of the tank, and a second connection arranged in the lower portion of the tank. Furthermore, said tank also has a biogas outlet and a lower surface configured to rest on a surface. Furthermore, the lower portion of the tank is at a shorter distance from the support surface compared to the upper portion of the tank.

Said organic matter biodigester also comprises a manual pump with a pump inlet connected in the second connection, and a pump outlet connected in the first connection of the tank. The pump is configured to recirculate organic matter within the tank from the second connection to the first connection and a porous layer arranged within the tank.

In particular, organic matter is introduced into the tank, which can be mixed with water. Said organic matter covers the porous layer inside the tank, allowing the bacteria contained by certain organic matter to increase the surface area inside the biodigester where the bacteria will be placed, thus increasing their cultivation area and, consequently, increasing the biodigester's performance. In addition, when the pump is activated, it causes the settled organic matter to flow from the bottom of the tank to the upper portion of the tank, thus stimulating the movement of bacteria within said tank.

### BRIEF DESCRIPTION OF THE DRAWINGS

FIG. 1 illustrates an isometric view of a biodigester including a tank with two connections, an organic matter inlet, and a biogas outlet, wherein the connections go to a manual pump. Said FIG. 1 further illustrates a vessel and a filter, both connected to the biogas outlet, and a detailed view of the manual pump.

FIG. 2 illustrates a side view of the biodigester of FIG. 1.

FIG. 3 illustrates another isometric perspective of the biodigester of FIG. 1, wherein said biodigester includes a control unit connected to the tank and a pipe orthogonally connected on one side of the tank.

FIG. 4 illustrates a side view of the biodigester of FIG. 1, wherein said biodigester is connected to another biodigester through a conduit.

### DETAILED DESCRIPTION OF THE INVENTION

The present disclosure relates to an organic matter biodigester, comprising a tank (1) having an organic matter inlet (5), a first connection (2) arranged in an upper portion (4) of the tank (1) and a second connection (3) arranged in the lower portion of the tank (1). Furthermore, said tank (1) also has a biogas outlet (6) and, optionally, a lower surface configured to rest on at least one support surface (7). Also, the lower portion of the tank (1) is at a shorter distance from the support surface (7) compared to the upper portion (4) of the tank (1).

Said organic matter biodigester also comprises a manual pump (8) with a pump inlet (8a) connected in the second connection (3), and a pump outlet (8b) connected in the first connection (2) of the tank (1). The pump (8) is configured to recirculate organic matter within the tank (1) from the second connection (3) to the first connection (2); and a porous layer (9) arranged within the tank (1).

In particular, organic matter, which can be mixed with water, is introduced into the tank (1) through the organic matter inlet. In addition, said organic matter totally or partially covers the porous layer (9) inside the tank (1), allowing the bacteria contained by certain organic matter to increase their cellular concentration, immobilizing on the surface of the porous layer (9) inside the biodigester where the bacteria will be placed, thus increasing the amount of bacteria and, consequently, the biodigester's performance. Furthermore, when the pump (8) is activated, it makes the settled organic matter flow from the second connection (3) to the first connection (2) located at the upper portion of the tank, thus promoting the movement of bacteria within said tank (1).

At least biogas and fertilizer are identified as products resulting from the process carried out in the biodigester. As biogas is a fuel, it can be used for cooking, generating electricity, or other gas appliances (refrigerators, radiators, lights, rice cookers, etc.).

For the purpose of the present disclosure, “organic matter” means those elements or substances that come from plants, trees, and animal waste, among others. That is, everything that comes from living organisms is biomass. Biomass is also produced through, for example, firewood; coffee residues; branches, bark, and sawdust; animal manure; sewage; organic waste or crops planted specifically to produce biomass, among others.

As for the process, the decomposition of organic matter inside the tank (1) occurs due to the action of at least four types of bacteria under anaerobic conditions. The first type are hydrolytic bacteria, which produce acetic acid, monocarbon compounds, organic fatty acids, and other polycarbonate compounds. The second type are acetogenic bacteria, which produce hydrogen. The third type are homoacetogenic bacteria, which transform multicarbon compounds into acetic acid. Finally, there are methanogenic bacteria, which produce methane (CH₄) in a ratio of about 40 to 70%, carbon dioxide (CO₂) in a ratio of 30 to 60%, hydrogen (H₂) from 0 to 1% and hydrogen sulfide gas (H₂S) from 0 to 3%.

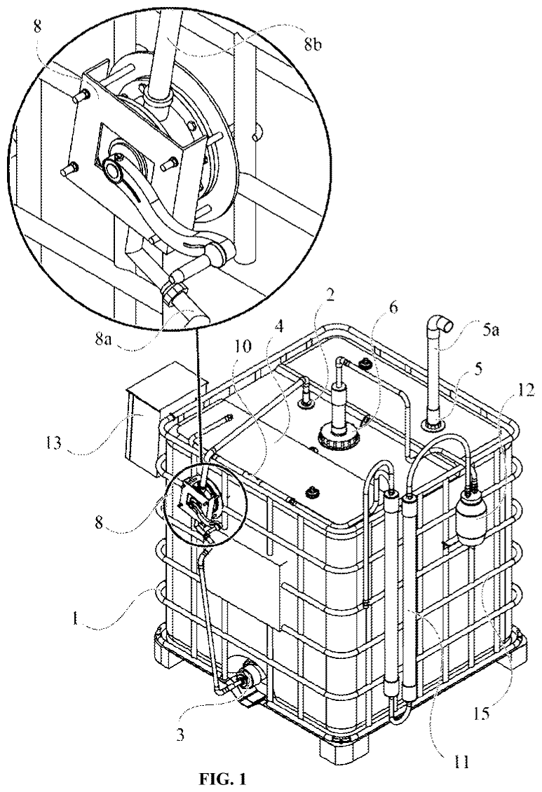

Referring to FIG. 1, the tank (1) has an organic matter inlet (5), a first connection (2) arranged in an upper portion (4) of the tank (1) and a second connection (3) arranged in the lower portion of the tank (1), wherein said connections (2, 3) are perforations of the tank. Said tank (1) further comprises a biogas outlet (6) configured to allow the biogas contained in said tank (1) to exit therefrom. And a bottom surface configured to rest on a support surface (7), wherein the lower portion of the tank (1) is at a shorter distance from the support surface (7) compared to the upper portion (4) of the tank (1). Furthermore, the organic matter inlet (5) can also be the outlet through which the organic matter contained in the tank (1) is removed, or the tank (1) can also have an organic matter outlet (14).

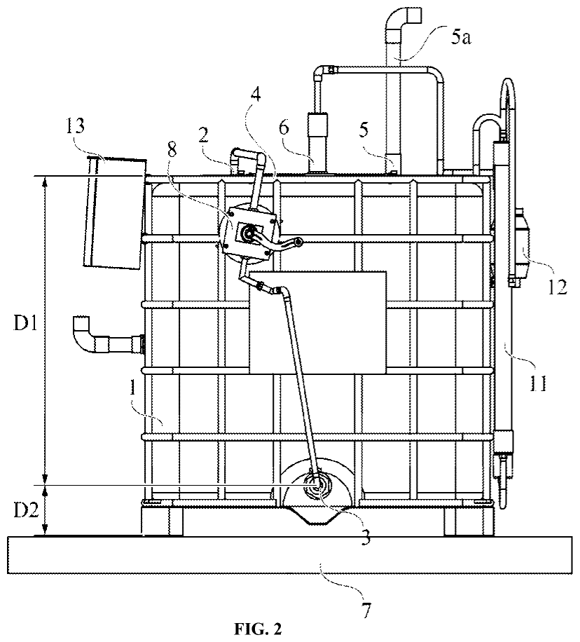

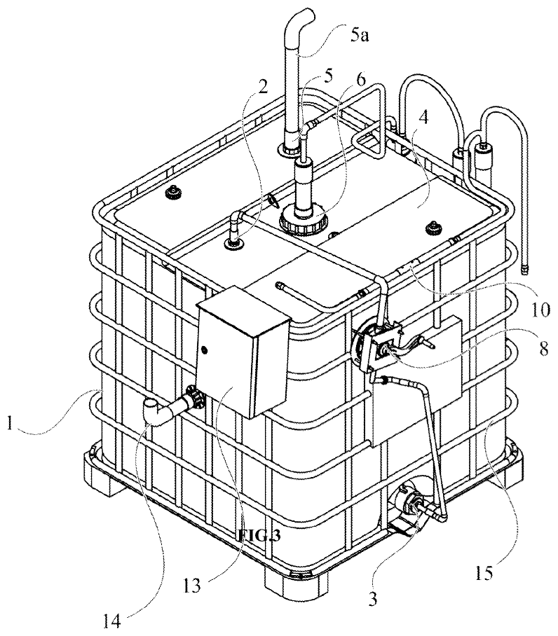

Referring to FIG. 2 and FIG. 3, the tank (1) is a tank with a cubic shape resting on four supports. Said four supports are in contact with a support surface (7) and, in addition, the first connection (2) is at of the support surface (7), while the second connection (3) is at a second distance (D2) from the support surface (7). Said first distance (D1) has a greater length than the second distance (D2), which allows the organic matter pumped by the pump (8) to move from the first connection (2) towards the porous layer (9).

Referring to FIG. 2, the fact that said tank (1) has a first connection (2) arranged in an upper portion (4) of the tank (1) and a second connection (3) arranged in the lower portion of the tank (1) allows the pump (8) to pump the organic matter arranged in the bottom of the tank (1), from the second connection (3) to the first connection (2). That is, said organic matter is transported from the bottom of the tank (1) to the upper portion (4) of the tank (1), allowing the organic matter to be stirred.

On the other hand, the tank (1) has an organic matter inlet (5), which can be in the upper portion (4), and a vertical pipe (5b) can also be connected there, arranged from the upper portion (4) of the tank (1) to the height of the biogas outlet (6) of the tank (1). Said vertical pipe (5b) allows the organic matter to move by gravity to the tank (1), which reduces the probability of any type of clogging with organic material. In addition, the height of the vertical pipe (5b) allows for a higher pressure gradient, which pushes the effluent out of the tank (1), based on the principle of communicating vessels. For example, when removing biomass located inside the tank (1) is desired and it comprises an organic matter outlet (14), introducing new organic matter through the vertical pipe (5b) connected at the organic matter inlet (5) increases the hydrostatic pressure generated inside the tank (1) which allows the organic matter previously stored in the tank to leave the tank (1) through said organic matter outlet (14).

To understand the present disclosure, the vertical pipe (5b) is understood to be vertical, being orthogonal to the support surface (7).

Optionally, the tank (1) can be covered by thermal insulation. This allows preserving the internal temperature and reducing the growth of oxygen-producing algae, which affect the anaerobic process. For example, said tank (1) may be insulated with polyethylene foams, greenhouses, matte black paint or matte black paint with additives, or any other insulation known to a person of ordinary skill in the art.

On the other hand, the material of the tank (1) is selected from the group consisting of polyvinyl chloride (PVC), chlorinated polyvinyl chloride (CPVC), polyethylene terephthalate (PET), (PA), (e.g., PA12, polyamide PA6, PA66); polychlorotrifluoroethylene (PCTFE); polyvinylidene fluoride (PVDF), reinforced with fibers (e.g., glass, aramid, polyester), polyethylene tetrafluoride (PTFE); ethylene-chlorotrifluoroethylene (ECTFE); plastics (polyester, vinylester, epoxy, vinyl resins), equivalent materials known to a person of ordinary skill in the art or a combination of the above.

The pump (8), for its part, is a manual pump (8) with a pump inlet (8a) connected in the second connection (3), and a pump outlet (8b) connected in the first connection (2) of the tank (1). As previously mentioned, said pump (8) allows a user to recirculate organic matter within the tank (1), which stirs said organic matter within the tank (1). The stirring allows the biological conditions inside the tank (1) to be uniform, which reduces sedimentation or stratification phenomena. This type of recirculation is not like the traditional type, taken from the lower portion to the upper portion. In the present disclosure, the microorganisms placed in the upper portion are taken and, from there, the pump inlet is taken, thus injecting it through the lower portion where the substrate is sedimented.

Furthermore, the pump (8) being manual means that there are no elements in the internal part of the biodigester that could break down or that require electrical energy to carry out the stirring.

Said pump (8) is selected from the group consisting of positive displacement manual pumps, manual diaphragm pumps, rotary pumps, equivalent pumps known to a person of ordinary skill in the art, or a combination thereof.

On the other hand, the porous layer (9) arranged inside the tank (1) can cover between 10% and 20%, between 10% and 60% of the internal volume of the tank (1), or it can cover no more than 60% of the inside of the tank (1). The porous layer (9) covering no more than 60% of the interior of the tank (1) allows the gases resulting from the reaction produced within said tank (1) to be stored in the remaining 40% of the internal space of the tank (1). Similarly, there is another free space with organic material and water. Also, the porous layer's presence keeps the biodigester's weight from significantly increasing.

The porous layer (9) is selected from the group consisting of a plurality of conduits, a plurality of PET conduits, and a plurality of PET cylinders with a diameter between 3 cm and 10 cm, between 5 cm and 9 cm, between 4 cm and 8 cm, between 3 cm and 7 cm, and a length between 3 cm and 10 cm, between 5 cm and 9 cm, between 4 cm and 8 cm, between 3 cm and 7 cm. Also, in one embodiment, said porous layer is composed of sections of recycled PET plastic bottles.

For example, the porous layer (9) may comprise a plurality of PET cylinders with a diameter between 3 cm and 10 cm and a length between 3 cm and 10 cm. This allows increasing the biogas production yield inside the tank (1) as it increases the surface area inside the biodigester, which, in turn, increases the bacteria cultivation area or cell immobilization inside the tank (1). This also allows increasing the residence times of microorganisms, since there is a greater contact surface where the bacteria can attach. On the other hand, the porous layer (9) having the previous configuration allows air and other gases to circulate more easily in the open spaces of the cylinders, compared to other possible conduits of smaller diameter, which encourages growth of bacteria.

A reservoir can be connected to the biogas outlet (6), which allows storing the biogas produced inside the tank (1) for future use. On the other hand, the biogas outlet (6) can also be connected to a heating medium, such as a gas appliance to use the biogas from the biodigester. Said reservoir can be an element of variable volume, such as a tire or another element made of an elastomeric material whose volume increases when filled with a fluid, in this case, biogas.

On the other hand, a flow meter (10) can be connected to the biogas outlet (6) of the tank (1), which is connected to a gas filtration system (11) and a vessel with a sodium hydroxide solution (12). Referring to FIG. 2 and FIG. 3, the biogas leaves the biogas outlet (6) of the tank (1) and goes through a gas filtration system (11), which allows impurities to be removed from the gas obtained. The gas is then moved through a vessel with a sodium hydroxide solution (12), which removes excess moisture from the biogas and also removes impurities that were not removed by the gas filtration system (11). Afterwards, the biogas passes through a flow meter (10), which allows obtaining data corresponding to the biogas flow.

In particular, the gas filtration system (11) can be a conduit through which the biogas circulates, which contains activated carbon and iron filings. For its part, the vessel with the sodium hydroxide solution (12) is a container with liquid sodium hydroxide, connected to a double-ended conduit, one end connected to the biogas outlet (6) of the tank (1) and another end immersed in sodium hydroxide solution. This allows the biogas to pass through the liquid sodium hydroxide and then move to the gas filtration system (11).

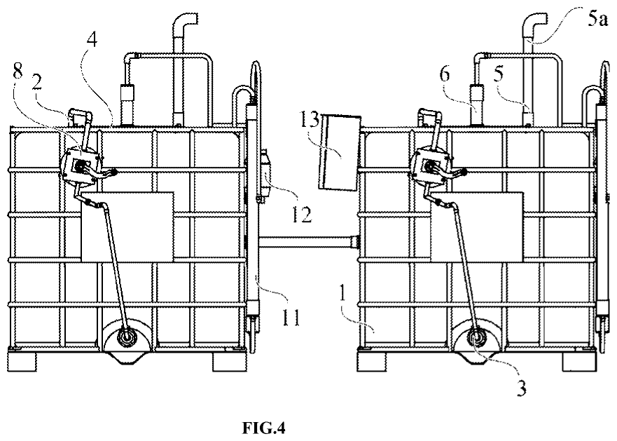

Referring to FIG. 4, and in one embodiment of the disclosure, two organic matter biodigesters are connected, where said biodigesters have the same elements as the biodigester of FIG. 3. On the other hand, said biodigesters are connected through a conduit connected between the tanks (1).

On the other hand, a pressure sensor configured to obtain pressure data inside the tank (1) and a temperature sensor configured to obtain temperature data inside the tank (1) can be arranged inside the tank (1). In addition, the flow meter (10), the pressure sensor, and the temperature sensor can be connected to a control unit (13).

In particular, the pressure data, the temperature data, and the flow data are sent to the control unit (13) and may be stored in a memory module of the control unit.

Said control unit (13) can be selected from the group consisting of: programmable logic controllers (PLC), microprocessors, DSCs (Digital Signal Controller), FPGAs (Field Programmable Gate Array), CPLDs (Complex Programmable Logic Device), ASICs (Application Specific Integrated Circuit), SoCs (System on Chip), PsoCs (Programmable System on Chip), computers, servers, tablets, cell phones, smartphones, signal generators and equivalent control units known to a person of ordinary skill in the art and combinations thereof.

In addition, the memory module of the control unit can be selected between RAM (cache memory, SRAM, DRAM, DDR), ROM (Flash, Cache, hard drives, SSD, EPROM, EEPROM, removable ROM (e.g., SD (miniSD, microSD, etc.), MMC (MultiMedia Card), Compact Flash, SMC (Smart Media Card), SDC (Secure Digital Card), MS (Memory Stick), among others), CD-ROM, digital versatile disks (DVD (Digital Versatile Disc)), or other optical storage, magnetic cassettes, magnetic tapes, storage or any other medium that can be used to store information and that can be accessed with the control unit. Instructions, data structures, and computer program modules are generally incorporated into memory registers. Some examples of data structures are: a text sheet or a spreadsheet, a database.

The control unit (13) may be connected to a display panel configured to show pressure, temperature and biogas flow information from the tank (1). Particularly, the data obtained by the pressure, temperature and flow sensors are sent to the control unit (13) and, then, said control unit (13) is configured to display said obtained data on a display panel so that a user can observe them.

Said display panel is selected from the group consisting of: CRT screen, VGA screen, SVGA screen, Plasma screen, LCD screen, LED screen, TouchScreen, or MultiTouch screen.

Furthermore, the display panel may be connected to a second control unit (13b) at a distance greater than one meter from the tank (1). Said control unit (13) and the second control unit (13b) may be connected by a wireless communications module, which uses a wireless communication technology that is selected from the group consisting of Bluetooth, WiFi, RF ID (Radio Frequency Identification), UWB (Ultra-Wide Band), GPRS, Konnex or KNX, DMX (Digital MultipleX), WiMax, and equivalent wireless communication technologies known to a person of ordinary skill in the art, and combinations thereof.

This allows the information obtained by the pressure, temperature and flow sensors to be sent remotely to the display panel so that a user can see the behavior inside the tank (1) without having to be near it.

### EXAMPLES

### Example 1

An organic matter biodigester was developed, comprising:
a tank (1) having an organic matter inlet (5), a first connection (2) arranged in an upper portion (4) of the tank (1) and a second connection (3) arranged in the lower portion of the tank (1). Said tank (1) also includes a biogas outlet (6) and a lower surface configured to rest on a support surface (7). Said lower portion of the tank (1) is at a shorter distance from the support surface (7) compared to the upper portion (4) of the tank (1);
a vertical pipe (5b) connected to the organic matter inlet (5) of the tank (1);
a pump (8), which is a manual rotary drum pump with a pump inlet (8a) connected in the second connection (3), and a pump outlet (8b) connected in the first connection (2) of the tank (1);
a porous layer (9) corresponding to a plurality of PET cylinders with a diameter between 3 cm and 10 cm and a length between 3 cm and 10 cm, arranged inside the tank (1).

In Example 1, the anaerobic digester was stirred manually, the organic matter was biodegraded, and 20% more biogas was produced. In addition, there was evidence of a 60% increase in temperature inside the reactor compared to the outside temperature. All data were obtained thanks to the variable control and monitoring system.

### Example 2

A matter biodigester was developed like that of Example 1, further including:
a flow meter (10) connected to the biogas outlet (6) of the tank (1);
which is connected to a system, both connected to said flow meter (10);
a pressure sensor and a temperature sensor, both arranged inside the tank (1);
a temperature sensor arranged outside the tank (1);
a control unit (13) connected to the flow meter (10), the pressure sensor, and the temperature sensor; and
a display panel
said control unit (13) is connected to a display panel.

In Example 2, it a user was able to monitor the variables of the tank (1), and the biogas produced inside it, from any geographical location with an internet connection and with any protocol. The user was able to monitor minute by minute the internal and external temperature of the tank (1), the biogas flow, and the pressure in the biogas storage tank.

It is to be understood that the present invention is not limited to the embodiments described and illustrated herein, for, as will be evident to a person of ordinary skill in the art, some possible variations and modifications do not depart from the spirit of the invention, which is only defined by the following claims.

1. An organic matter biodigester, comprising:
a tank (1) having:
an organic matter inlet (5), a first connection (2) arranged in an upper portion (4) of the tank (1) and a second connection (3) arranged in the lower portion of the tank (1);
a biogas outlet (6); and
a lower surface configured to rest on a support surface (7);
wherein the lower portion of the tank (1) is at a shorter distance from the support surface (7) compared to the upper portion (4) of the tank (1);
a manual pump (8) with a pump inlet (8a) connected in the second connection (3), and a pump outlet (8b) connected in the first connection (2) of the tank (1);
wherein the pump (8) is configured to recirculate organic matter within the tank (1) from the second connection (3) to the first connection (2); and
a porous layer (9) arranged inside the tank (1).

2. The biodigester of claim 1, wherein the porous layer (9) is selected from the group consisting of a plurality of conduits, a plurality of PET conduits, and a plurality of polyethylene terephthalate cylinders with a diameter between 3 cm and 10 cm and a length of between 3 cm and 10 cm.

3. The biodigester of claim 1, wherein the porous layer (9) covers no more than 60% of the interior of the tank (1).

4. The biodigester of claim 1, wherein the organic matter inlet (5) corresponds to a vertical pipe (5b) arranged from the upper portion of the tank (1) up to the height of the biogas outlet (6) of the tank (1).

5. The biodigester of claim 1, characterized in that a flow meter (10) is connected to the biogas outlet (6) of the tank (1); the flow meter (10) is connected to a gas filtration system (11) and a vessel with a sodium hydroxide solution (12).

6. The biodigester of claim 5, characterized in that the flow meter (10) is connected to a control unit (13) that connects to a display panel configured to show pressure, temperature, and biogas flow information from the tank (1).

7. The biodigester of claim 6, wherein a pressure sensor and a temperature sensor connected to the control unit (13) are arranged inside the tank (1).

8. The biodigester of claim 1, wherein the tank (1) is connected to a reservoir that allows storing the biogas produced within said tank (1).

9. The biodigester of claim 1, wherein the pump (8) is selected from the group consisting of rotary pumps, a manual rotary drum pump, positive displacement manual pumps, and manual diaphragm pumps.
